(19) United States
(12) Patent Application Publication
(10) Pub. No.: US 2005/0019207 A1
(43) Pub. Date: Jan. 27, 2005

(54) Process for deodorizing and disinfecting a bioorganic material

(76) Inventors: Michael John Basil Oliver, Pinetown (ZA); Milan Carsky, Pinetown (ZA)

Correspondence Address: JOHN S. PRATT, ESQ;KILPATRICK STOCKTON, LLP, 1100 PEACHTREE STREET, ATLANTA, GA, 30309, US

(21) Appl. No.: 10/494,231

(22) PCT Filed: Oct. 25, 2002
(86) PCT No.: PCT/IB02/04434

(30) Foreign Application Priority Data
Nov. 2, 2001 (ZA) 2001/9082

(51) Int. Cl. A61L 2/16

(52) U.S. Cl. 422/5; 422/28; 71/11

(57) ABSTRACT

The invention relates to a process for deodorizing and disinfecting a bioorganic material. The process provides for nitric acid to be formed by a displacement reaction between at least one nitrate salt and sulphuric acid, the nitric acid being mixed with the bioorganic material to be deodorized and disinfected. By forming the bioorganic material and nitric acid into a homogeneous paste having a pH below 2.5, the nitric acid will react oxidatively with the odorous components of the bioorganic material to form a chemically deodorized and disinfected reacted paste. The process can then be completed by neutralizing the reacted paste, which can hence be rendered suitable for forming a wet, organically enriched inorganic fertilizer mix that can be dried and pelletized into a compact particle fertilizer.

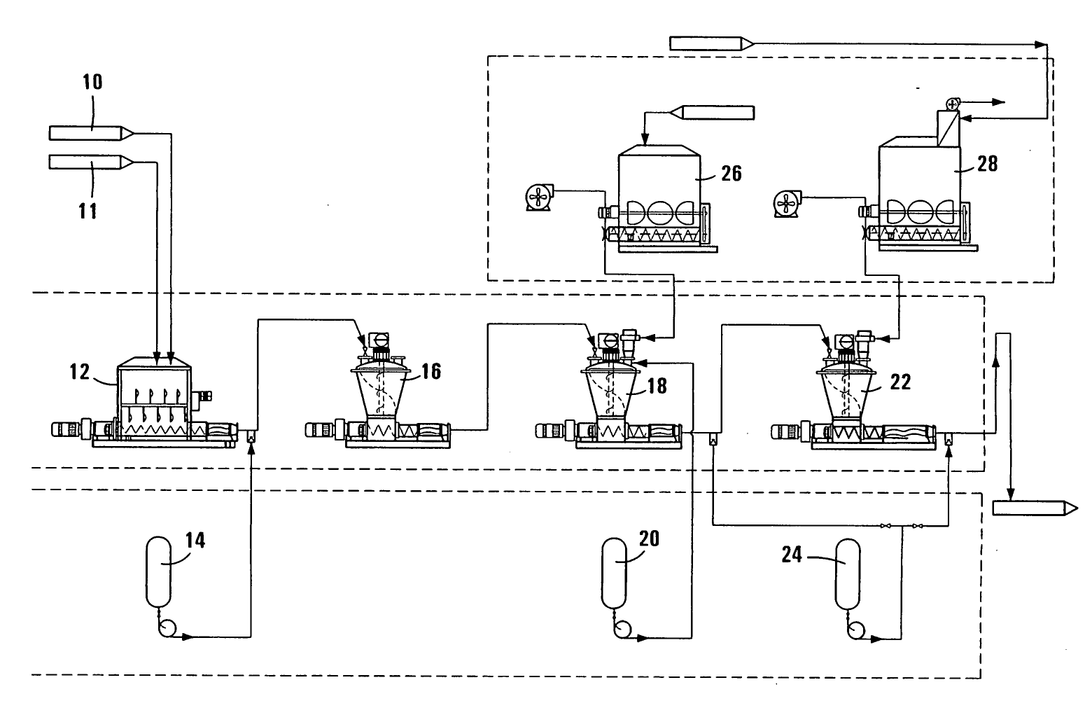

THIS INVENTION relates to a process for deodorizing and disinfecting a bioorganic material.

There exists increasing pressure to find an environmentally safe method of recycling bioorganic materials, regulations in many countries rapidly becoming more stringent with respect to disposal of such materials and tending towards the highest level of safety, which is termed “Exceptional Quality Standards”. Relevant criteria established require strict limits of pathogen reduction, vector attraction standards and heavy metal concentrations. When products do meet these standards, they require no further level of quality regulation and the products can thus be moved into commerce.

Typically in the United States, the United States Environmental Protection Agency (U.S. EPA) regulates the use of bioorganic materials, such a sewage sludge, in their natural form for land applications. As such, the present invention relates also to the use of such bioorganic materials, that form the subject matter of U.S. EPA regulations, as part of organically enriched inorganic compact fertilizers. Any reference hereinafter to a bioorganic material or to a biosolid material must be interpreted as such.

The EPA 40 CFR Part 503 standards include in particular criteria for biosolid disposal and category options for land application. Biosolids that meet the “Class A” pathogen reduction requirements in § 503.22(a) and the vector attraction reduction requirements in paragraph 503.33 (b)(1)-(b) (8), because of their low pollutant concentration and treatment to reduce pathogens to below detectable levels and reduced vector attraction, can be sold to the public without the restrictions required for land application of biosolids. The U.S. EPA also promulgated rules to treat waste water sludge containing odour, animal viruses, pathogenic bacteria, and parasites, which will permit use of the waste water sludge as a fertilizer for agricultural lands and application directly to land as a dry granular material.

The treatment of bioorganic materials, essentially to deal with U.S. EPA regulations, form at least part of the subject matter of various United States patents, including U.S. Pat. No. 4,781,842 (Nicholson), U.S. Pat. No. 4,902,431 (Nicholson et al), U.S. Pat. No. 5,013,548 (Christy et al), U.S. Pat. No. 5,1 35,664 (Burnham), U.S. Pat. No. 5,275,733 (Burnham), U.S. Pat. No. 5,417,861 (Burnham), U.S. Pat. No. 5,422,01 5 (Angell et al), U.S. Pat. No. 5,435,923 (Girovich) and U.S. Pat. No. 5,554,279 (Christy). However, the processes referred to are not considered suitable and/or economically feasible, particularly in relation to the production of a compact granular fertilizer formed from a biosolids material that must be converted to a fertilizer mix and then dried and pelletized.

International patent application PCT/IB01/00349 (Energy Engineering (Pty) Ltd et al) relates to the conversion of a bioorganic material into a wet, organically enriched inorganic fertilizer mix that can be dried and pelletized into a compact particle fertilizer and also deals particularly with U.S. EPA regulations. The treatment process involved requires the use of both nitric acid and ammonia and although the process as such deals essentially with U.S. EPA regulations, insofar as sewage works are normally located in densely populated areas, it is not practical to permit the use of these chemicals, with both storage and use being potentially dangerous.

It is thus an object of this invention to provide a simple environmentally acceptable process for deodorizing and disinfecting a bioorganic material and, as such, a fertilizer manufacturing process for converting sewage sludge, or a similar bioorganic material, into a deodorized, sterilized and enhanced organically enriched fertilizer mix, which can be dried and pelletized to form compact fertilizer granules, which process is cost effective and particularly also complies with or exceeds U.S. EPA regulations for the provision of at least “Class A” biosolid material and also “exceptional quality” product material, both in respect of the wet process and in respect of the dried product produced by the process.

Existing drying operations involving bioorganic materials are also plagued by potential fire hazards and it is thus a further object of this invention, in relation to the use of such bioorganic materials, to alleviate this problem in the drying process.

It is still a further object of this invention to produce an organically enriched inorganic fertilizer in a compact particle form that is suitable for distribution by conventional agricultural fertilizer spreaders.

According to the invention there is provided a process for deodorizing and disinfecting a bioorganic material, which includes the steps of
inducing a displacement reaction between at least one nitrate salt and sulphuric acid for forming nitric acid and mixing the nitric acid with the bioorganic material to be deodorized and disinfected to form a homogeneous paste therewith, until the paste has a pH below 2.5;
permitting, for a period of at least ten minutes, the nitric acid within the paste to react oxidatively with the odorous components of the bioorganic material for deodorizing the odorous components, to degrade non-keratin proteins to peptide fragments and amino acids, to effect microbial disinfection of the bioorganic material, to eliminate pathogens, viruses and bacteria within the bioorganic material and to react with trace elements to form nitrate complexes of these elements, thereby forming a chemically deodorized and disinfected reacted paste; and
after the formation of the reacted paste, neutralizing the reacted paste with an alkaline substance.

The at least one nitrate salt that is used within the displacement reaction that is induced may be at least of one potassium nitrate and ammonium nitrate. As such, the displacement reaction that is induced provides for the nitrate radicals in the potassium nitrate and/or ammonium nitrate to be displaced, thereby to form potassium sulphate and/or ammonium sulphate and nitric acid. The quantity of sulphuric acid used must be such that the prevalent pH is low enough to initiate the displacement reaction, a pH below 2.5 normally being suitable.

The nitric acid provided within the wet bioorganic material and formed by the displacement reaction may be formed before being mixed into the bioorganic material. Preferably, however, the process of the invention includes inducing the displacement reaction by mixing the at least one nitrate salt with the bioorganic material, adding the sulphuric acid and liquidizing the liquid mixture to form the substantially homogeneous paste and then permitting the displacement reaction to occur for forming the nitric acid.

The nitric acid that is displaced from the at least one nitrate salt may constitute at least 2% by mass of the bioorganic material.

The process of the invention may include permitting the nitric acid within the paste to react oxidatively with the odorous components of the bioorganic material for deodorizing the odorous components, to degrade non-keratin proteins to peptide fragments and amino acids, to effect microbial disinfection of the bioorganic material, to eliminate pathogens, viruses and bacteria within the bioorganic material and to react with trace elements to form nitrate complexes of these elements, for a period longer than 30 minutes.

The process of the invention may include neutralizing the reacted paste by adding di-ammonium phosphate to the paste to react with nitric acid residue, forming mono-ammonium phosphate and ammonia as an alkaline substance that neutralises the nitric acid by forming ammonium nitrate. The phosphate formed will provide the neutralized reacted paste, particularly when dried, with fire retardant properties.

Alternatively, or in addition, the process may include neutralizing the reacted paste by adding directly an alkaline substance, which is at least of calcium oxide, limestone and ammonia to the reacted paste and providing for the formation of nitrates as a result of remaining nitric acid being neutralized.

Neutralizing of the reacted paste provides for a pH adjustment of the reacted paste to a value between 5 and 7.

The process of the invention still further may include mixing into the reacted paste potassium compounds selected from a group including potassium chloride, potassium bicarbonate and urea potassium bicarbonate. Within a dried, deodorized and disinfected bioorganic material, the potassium compounds serve to enhance the fire retardant properties of the bioorganic material.

The process of the invention as above defined provides a deodorized and disinfected bioorganic material that can serve as a wet, organically enriched inorganic fertilizer mix that can be dried and pelletized into a compact particle fertilizer. In order to further enhance this application of the deodorized and disinfected bioorganic material, the process of the invention may include adding into the neutralized reacted paste inorganic fertilizer substances for providing a wet fertilizer mix having a desired nitrogen/phosphorus/potassium (NPK) composition. It is well known in this regard that fertilizers for different applications require different NPK compositions and the addition of the said additional inorganic fertilizer substances clearly can provide for a fertilizer to have a specific required NPK composition.

The process of the invention may thus provide for the product of the process to be formed to comprise a wet organically enriched inorganic fertilizer mix that can be dried and pelletized into a compact particle fertilizer. The process of the invention accordingly may form part of a process for producing an organically enriched inorganic fertilizer which includes, following the formation of a wet organically enriched inorganic fertilizer mix, in accordance with the process of the invention, sterilizing the mix and pelletizing the mix into a compact particle fertilizer by heating and drying the mix within a pelletizer apparatus.

Pelletizing of the wet organically enriched inorganic fertilizer mix into a compact particle form particularly may be carried out within a fluidized bed apparatus, or within a granulation drum.

The processes of the invention are described in more detail hereinafter, by way of example, with reference to the accompanying diagrammatic flow diagram which illustrates particularly a process for deodorizing and disinfecting a bioorganic material, particularly insofar as this process serves also for converting a bioorganic material into a wet organically enriched inorganic fertilizer mix that can be dried and pelletized into a compact particle fertilizer.

Referring to the flow diagram, the process for deodorizing and disinfecting a bioorganic material, particularly as part of a process for converting a bioorganic material into a wet organically enriched inorganic fertilizer mix that can be dried and pelletized into a compact particle fertilizer, in accordance with the first and second aspects of the invention, respectively, incorporates the feed of a bioorganic sludge material at a controlled rate from a supply source 10 to a mixer hopper 12, the hopper incorporating a displacement means for displacing the sludge therefrom. The bioorganic sludge material typically consists of sewage sludge.

The first step in the process provides for nitrate salts, in the form of potassium nitrate and ammonium nitrate, to be fed at a controlled rate into the mixer hopper 12, particularly from a supply source 11 and at the upstream end of the mixer hopper 12. The mixer hopper hence provides for effective mixing of the sludge material and the nitrate salts. The mixture is then displaced to a further mixer hopper 16, while sulphuric acid is simultaneously added thereto, from a supply vessel 14. Within the mixer hopper 16 the mixture from the hopper 12 and the sulphuric acid is liquidized to form a substantially homogeneous past, the quantity of sulphuric acid particularly being such that the pH of the mix is lowered to a level low enough to initiate a displacement reaction with the nitrate salts, a pH below 2.5 being normally required. By adding the sulphuric acid to the sludge material, the displacement reaction referred to is induced between the nitrate salts and the sulphuric acid, this displacement reaction displacing the nitrate radical in each of the potassium nitrate and the ammonium nitrate to form potassium sulphate and ammonium sulphate respectively, and nitric acid. The paste so formed must have sufficient nitric acid mixed therein to provide the paste with a pH below 2.5.

The nitric acid within the paste is then permitted to oxidatively react with the odorous components of the bioorganic material for deodorizing the odorous components, to degrade non-keratin proteins to peptide fragments and amino acids, to effect microbial disinfection of the bioorganic material, to eliminate pathogens, viruses and bacteria within the bioorganic material and to react with trace elements to form nitrate complexes of these elements, thereby forming a chemically deodorized and sterilized reacted paste. The reaction time for the above purpose within the hopper 16 preferably is at least thirty minutes.

The reacted paste that is formed is then displaced into a third mixing hopper 18, into which di-ammonium phosphate is fed at a controlled rate from a supply source 26, the di-ammonium phosphate serving to neutralize the oxidative reaction together with any other neutralizing fertilizer agents, as is necessary to achieve a pH between 5 and 7. Mono-ammonium phosphate provided through this neutralizing also will provide dried and pelletized compact particle fertilizer, formed as described hereafter, with required fire retardant properties. As an alternative to the use of di-ammonium phosphate for neutralizing the reacted paste, or in addition thereto, calcium oxide or limestone can be fed at a controlled rate from the hopper 26 into the mixing hopper 18, thus also reacting with the acids for neutralizing the paste.

Neutralizing of the reacted paste also may be carried out by feeding ammonia directly to the further reacted paste, this method of neutralizing being usable where ammonia is readily and safely available and where use of ammonia is permitted.

The process of the invention may optionally provide for phosphoric acid and/or sulphuric acid to be fed into the mixing hopper 18 prior to feeding the paste to a hopper 22, with ammonia being fed from a supply 24 to the reacted paste upstream of the hopper 22. The ammonia again serves to adjust the pH of the reacted paste, particularly to a value between 5 and 7. The above neutralizing process clearly provides for the formation of nitrates, sulphates and phosphates, as part of the process, the sulphates and phosphates also providing a dried and pelletized compact particle fertilizer, formed as described hereafter, with required fire retardant properties.

The addition of the sulphuric acid also can serve to oxidatively react with the odorous components of the bioorganic material for deodorizing the odorous components, to degrade non-keratin proteins to peptide fragments and amino acids, to effect microbial disinfection of the bioorganic material and to eliminate pathogens, viruses and bacteria within the bioorganic material, particularly insofar as the nitric acid may not have dealt with the above completely.

Additional dry chemical substances including any one of or a combination of di-ammonium phosphate, potassium chloride, potassium sulphate, ammonium sulphate and calcium oxide can be fed from the hopper 26 into either hopper 18 or 22, whereas dry chemicals including potassium bicarbonate, urea potassium bicarbonate, and other inorganic fertilizer substances can be mixed at a controlled rate into the neutralized reacted paste from a hopper 28, particularly to provide the final mix that is formed with a require NPK composition, as determined by the requirements of the fertilizer to be produced.

It will be appreciated that the process of the invention as described above is greatly variable while still incorporating the essential principles of the invention, which provide for the addition of nitrate salts, in the form of potassium nitrate and ammonium nitrate, to the bioorganic material being treated and permitting sulphuric acid to react with the nitrate salts by displacing the nitrate radicals in the salts to form potassium sulphate and ammonium sulphate respectively, and nitric acid, which serves to chemically deodorize and disinfect the bioorganic material.

The final mix can hence be conveyed to a dryer and pelletizer which, typically, is a fluidized bed-type dryer, in order to form a sterilized dried and pelletized compact particle fertilizer of a desired particle size. The complete process for forming a dry compact particle fertilizer thus incorporates the process of converting a bioorganic material into a wet organically enriched inorganic fertilizer mix, as above described, and illustrated in the flow diagram, together with the subsequent drying and pelletizing stage in order to form the required fertilizer product.

This process may provide also for recycled dust from the dryer used to be fed back to the final mix via the hopper 28 to the mixing hopper 22, thus further enhancing the drying and pelletizing process.

Referring to the above process as described and particularly the reactions of the bioorganic material with particularly nitric acid and optionally sulphuric acid, disinfection of the bioorganic material will occur as a result of protein destruction by the acids and the formation of peptide fragments and amino acids. This disinfection process is already discussed and, as such, is not described in further detail herein. Final sterilization of the product being produced in fact occurs during drying and pelletizing, which occurs at suitably elevated temperatures for required time periods and also through dehydration of microorganism cellular structures at these temperatures.

It must be appreciated that the apparatus associated with the process and as illustrated within the flow diagram is greatly variable and that the individual hoppers utilized can be particularly adapted to induce required reactions, while the various feed mechanisms can provide for the feed of both dry and wet materials into hoppers at controlled rates, thus permitting a substantially continuous process.

It must also be understood that the specific process steps as defined can be varied in various different respect while still incorporating the essential features of the process steps as hereinabove described and defined, and the invention extends also to variations of the process of the invention which incorporate such alternative process steps.

A compact particle fertilizer having as a major component a bioorganic material and formed in accordance with the invention will constitute an economically produced product, which complies with and generally exceeds U.S. EPA regulations, the compact particle fertilizer constituting at least a “Class A” fertilizer and generally also an “exceptional quality” fertilizer. It will be understood that this fertilizer process is carried out without the use of chemical substances that can be considered dangerous in relation to storage and use, e.g. nitric acid and ammonia.

By pelletizing the final mix formed in a fluidized bed-type drying and pelletizing apparatus, the compact particles formed will have a particle size range that is sufficiently constant to permit distribution by conventional agricultural fertilizer spreaders, which clearly renders use of such fertilizers practical and cost effective.

Insofar as different NPK compositions may be required for different fertilizer applications, it will be appreciated that the process of the invention conveniently accommodates this requirement by permitting the feed of dry inorganic fertilizer substances to be mixed into the organically enriched inorganic fertilizer mix, prior to its formation into a particle form.

The table below identifies the common odorants in bioorganic materials and their reactions with nitric acid and sulphuric acid respectively, thus further clarifying the process of the invention as hereinabove described.

Common Odorants in Bio-Solids:

Odor control Reactions

| | Nitric Acid | Sulphuric Acid |
|---|---|---|
| Sulphur Compounds | | |
| Dihydrogen sulphide | H₂S + 2HNO₃ = 4H₂O + S + 2NO | H₂S + H₂SO₄ = S + 2H₂O + SO₂ |
| Carbon disulphide | CS₂ + 4HNO₃ = CO₂ + 2S + 4NO₂ + 2H₂O | CS₂ + 2H₂SO₄ = CO₂ + 2S + 2SO₂ + 2H₂O |
| methyl mercaptan: | CH₃SH + 2HNO₃ = H₂O + CH₃SO₃H + 2NO | |
| Dimetyldisulphide | 3CH₃SCH₃ + 4HNO₃ = 3CH₃SO₂CH₃ + 4NO + | |
| | 2H₂O | |
| Nitrogen compounds | | |
| Ammonia | NH₃ + HNO₃ = NH₄NO₃ | 2NH₃ + H₂SO₄ = (NH₄)₂SO₄ |
| Trimethylamine: | (CH₃)₃N + HNO₃ = (CH₃)₃NHNO₃ | (CH₃)₃N + H₂SO₄ = (CH₃)₃NH₂SO4 |
| Trimethylamine: | (C₂H₅)₃N + HNO₃ = (CH₃)₃NHNO₃ | (C₂H₅)₃N + H₂SO₄ = (CH₃)₃NH₂SO4 |
| 2-butamine | CH₃—CHNH₂—CH₂—CH₃ + HNO₃ = | CH₃—CHNH₂—CH₂—CH₃ + H₂SO₄ = |
| | CH₃—CHNH₃NO₃—CH₂—CH₃ | CH₃—CHNH₃HSO₄—CH₂—CH₃ |
| Acetonitrile | CH₃CN + 2H₂O = CH₃COOH + NH₃ (acid | |
| | hydrolysis) | |
| Alcohols & ketones | | |
| Methanol: | CH₃OH + HNO₃ = H₂O + CH₃NO₃ | 2CH₃OH + H₂SO₄ = 2H₂O + (CH₃O)₂SO₂ |
| ethanol: | C₂H₅OH + HNO₃ = H₂O + C₂H₅NO₃ | 2C₂H₅OH + H₂SO₄ = 2H₂O + (C₂H₅O)₂SO₂ |
| butanol | C₄H₉OH + HNO₃ = H₂O + C₄H₉NO₃ | 2C₄H₉OH + H₂SO₄ = 2H₂O + |
| | | C₄H₉O—SO₂—O C₄H₉ |
| acetone: | CH₃—CO—CH₃ + 4HNO₃ = CH₃COOH + | |
| | 2NO + 2NO₂ + 3H₂O + CO₂ | |
| methyl-ethyl ketone: | CH₃—CO—CH₂CH₃ + 2HNO₃ = | |
| | 2CH₃COOH + 2NO + H₂O | |
| 3-methyl-pentanal: | CH₃—CH₂—CHCH₃—CH₂CHO + | |
| | 2HNO₃ = CH₃—CH₂—CHCH₃—CH₂COOH + | |
| | 2NO₂ + H₂O | |
| Other odorants: | | |
| Acetaldehyde: | CH₃CHO + 2HNO₃ = CH₃COOH + 2NO₂ + | |
| | H₂O | |
| heptanal | CH₃(CH₂)₅CHO + 2HNO₃ = | |
| | CH₃(CH₂)₅COOH + 2NO₂ + H₂O | |
| Pentane | C₅H₁₂ + HNO₃ = C₅H₁₁NO₂ + H₂O | |
| hexane | C₆H₁₄ + HNO₃ = C₆H₁₃NO₂ + H₂O | |
| octane | C₈H₁₈ + HNO₃ = C₈H₁₇NO₂ + H₂O | |

1. A process for deodorizing and disinfecting a bioorganic material, which includes the steps of
inducing a displacement reaction between at least one nitrate salt and sulphuric acid for forming nitric acid and mixing the nitric acid with the bioorganic material to be deodorized and disinfected to form a homogeneous paste therewith, until the paste has a ph below 2.5;
permitting, for a period of at least ten minutes, the nitric acid within the paste to react oxidatively with the odorous components of the bioorganic material for deodorizing the odorous components, to degrade non-keratin proteins to peptide fragments and amino acids, to effect microbial disinfection of the bioorganic material, to eliminate pathogens, viruses and bacteria within the bioorganic material and to react with trace elements to form nitrate complexes of these elements, thereby forming a chemically deodorized and disinfected reacted paste; and
after the formation of the reacted paste, neutralizing the reacted paste with an alkaline substance:

2. A process as claimed in claim 1, in which the at least one nitrate salt is at least one of potassium nitrate and ammonium nitrate.

3. A process as claimed in claim 1, which includes inducing the displacement reaction by mixing the at least one nitrate salt with the bioorganic material, adding the sulphuric acid and liquidizing the liquid mixture to form the substantially homogeneous paste and then permitting the displacement reaction to occur for forming the nitric acid.

4. A process as claimed in claim 1, in which the nitric acid displaced from the at least one nitrate salt constitutes at least 2% by mass of the bioorganic material.

5. A process as claimed in claim 1, which includes permitting the nitric acid within the paste to react oxidatively with the odorous components of the bioorganic material for deodorizing the odorous components, to degrade non-keratin proteins to peptide fragments and amino acids, to effect microbial disinfection of the bioorganic material, to eliminate pathogens, viruses and bacteria within the bioorganic material and to react with trace elements to form nitrate complexes of these elements, for a period longer than thirty minutes.

6. A process as claimed in claim 1, which includes neutralizing the reacted paste by adding di-ammonium phosphate to the paste to react with nitric acid residue, forming mono-ammonium phosphate and ammonia as an alkaline substance that neutralises the nitric acid by forming ammonium nitrate.

7. A process as claimed in claim 1, which includes neutralizing the reacted paste by adding directly an alkaline substance, which is at least one of calcium oxide, lime stone and ammonia, to the reacted paste and providing for the formation of nitrates as a result of remaining nitric acid being neutralized.

8. A process as claimed in claim 1, in which neutralizing of the reacted paste provides for a pH adjustment of the reacted paste to a value between 5 and 7.

9. A process as claimed in claim 1, which includes mixing into the reacted paste potassium compounds selected from a group including potassium chloride, potassium bicarbonate and urea potassium bicarbonate.

10. A process as claimed in claim 1, which includes adding into the neutralized reacted paste inorganic fertilizer substances for providing a wet fertilizer mix having a desired nitrogen phosphorus/potassium composition,

11. A process as claimed in claim 1, in which the product of the process is formed to comprise a wet organically enriched inorganic fertilizer mix that can be dried and pelletized into a compact particle fertilizer.

12. A process for producing an organically enriched inorganic compact particle fertilizer, which includes
forming a wet organically enriched inorganic fertilizer mix, in accordance with the process for deodorizing and disinfecting a bioorganic material as claimed in claim 11; and
sterilizing the mix and pelletizing the mix into a compact particle fertilizer by heating and drying the mix within a pelletizer apparatus.

13. A process for deodorizing and disinfecting a bioorganic material substantially as described in the specification.
